(19) United States
(12) Patent Application Publication
(10) Pub. No.: US 2015/0208934 A1
(43) Pub. Date: Jul. 30, 2015

(54) Method And Apparatus For The Detection Of Neural Tissue

(71) Applicants: Genevieve Sztrubel, Ramat Hasharon (IL); Arnaldo Mayer, Ramat Hasharon (IL)

(72) Inventors: Genevieve Sztrubel, Ramat Hasharon (IL); Arnaldo Mayer, Ramat Hasharon (IL)

(21) Appl. No.: 14/163,142

(22) Filed: Jan. 24, 2014

(51) Int. Cl. A61B 5/04; A61B 5/00

(52) U.S. Cl. CPC A61B 5/04001; A61B 5/0048

(57) ABSTRACT

The invention relates to a method and apparatus for neural tissue detection carried out using the Neural Tissue Detector (NTD), which is the apparatus that embodies the hardware aspect of the invention. The NTD enables neural tissue detection by stimulating a small tissue area and by measuring possible occurrences of induced response from neural tissue, if neural tissue is present in said small stimulated tissue area. The information gathered by the measurement provides a real-time assessment of the nature of the tissue which is targeted by the NTD. The invention is applicable to all types of neural tissues, including motor, and/or sensory and/or other types of neural tissues. The invention offers particular advantages in robot based surgical procedures or intravascular catheter based procedures but can also be used for manual surgery, according to different specific embodiments.

### FIELD OF THE INVENTION

The invention disclosed in this patent application relates to the field of electrophysiology and, more specifically, to the field of neural tissue detection. The invention relates to a variety of uses and applications for said neural tissue detection, including neural tissue sparing or neural tissue denervation.

This invention relates to a novel method and apparatus for neural tissue detection carried out using the Neural Tissue Detector (NTD), which is the apparatus that embodies the hardware aspect of the invention, described hereafter in this patent application.

The NTD enables neural tissue detection by stimulating a small tissue area and by measuring possible occurrences of induced response from neural tissue, if neural tissue is present in said small stimulated tissue area.

The information gathered by said measurement provides a real-time assessment of the nature of the tissue which is targeted by the NTD.

The invention is applicable to all types of neural tissues, including motor, and/or sensory and/or other types of neural tissues.

The invention offers particular advantages in robot based surgical procedures or intravascular catheter based procedures but can also be used for manual surgery, according to different specific embodiments.

Before we proceed, it is stressed that in this application, the term “neural tissue” relates and will be used equivalently to one or more of the following: neural cells, neurons of the central and/or peripheral nervous systems, bundles of said neurons, e.g. nerves, neural tissue cells, and/or any kind of tissue embedding any of the previous items.

It is further stressed that in this application the term “targeted area” or “targeted tissue” may be used interchangeably and relates to the area enclosed in a specific position within the area of interest in patient's body that the NTD can stimulate using a single emitter or a number of emitters—as explained hereafter—while trying to detect the presence of neural tissue in the patient's body.

The targeted area may be seen as the resolution afforded by the specific technology adopted in a certain implementation, while the term “area of interest” refers to a part of the patient's body for which information regarding the presence of neural tissue is sought. Consequently, the whole area of interest in the patient's body, while using the NTD, may be regarded as a collection of candidate targeted areas.

Of course it is stressed that the term “area” both in term “targeted area” and “area of interest” does not relate merely to a bi-dimensional surface but should be construed as meaning the upper face of a tridimensional piece of tissue where the presence of neural tissue is assessed. This is self evident since body sections where neural tissue may be present and the neural tissue itself are by essence tridimensional. Furthermore the term “area” refers to a tridimensional piece of tissue the upper surface of which may be located at any depth of the patient's body. The depth of the targeted area depends on the technology employed in a specific embodiment and to way this technology is applied in the course of the procedure.

Said area of interest in the patient's body may be inspected merely for the purpose of detecting the presence of neural tissue within it or other medical or surgical steps, may be added as the inspection proceeds, or after the inspection's completion. Obviously, at least in light of existing technologies and in the foreseeable future, in order to cover all said area of interest in the patient's body, the NTD will have to be used repeatedly in the course of the complete inspection of the area of interest.

### BACKGROUND OF THE INVENTION AND PRIOR ART

In general surgical practice, the problem of neural tissues sparing is well known.

Often, carrying out a surgical procedure entails damaging neural tissue in the operated area, for example, during the removal of a tumor. A well known example of such a risk is exemplified in the course of radical prostatectomy.

The surgical team who carries out the operation faces the challenge of recognizing neural tissue and avoiding harming it in the course of tumor resection.

Since the distinction between the neural tissue and tissue to be removed is generally carried out by visual inspection of the medical team, there is a significant risk of harming neural tissue.

In the event of such damage, post operatory consequences like, for instance, erectile dysfunction would be caused.

In the field of brain surgery, post operatory morbidity resulting from neural tissue damage such as speech, visual, and motor impairment are also well known.

Even in the field of elective surgery such as plastic surgery, iatrogenic visual impairment may occur as a result of neural tissue damage.

At this point, it may be useful to make two observations regarding the above mentioned risks: neural tissue may be difficult to distinguish because of the small dimension of the embedded nerve network. Even if the neural tissue is successfully identified the spatial density of the embedded nerve network and its proximity to the tissues which are in the area subject to the surgery, make it technically difficult to avoid neural tissue harming.

A number of solutions to the problems discussed above have been suggested.

In U.S. Pat. No. 7,720,532 B2 a manually held tool is provided, having a tissue-type and distance-measuring sensor. Both sensors are used in conjunction to verify the presence of a “safe margin” distance between a first tissue type zone (typically a tumor) and a second tissue type zone (typically healthy tissue) located on the incision path of the resection tool. In case the safe margin presence is not verified, the tool may induce a deviation of the incision path in order to verify the “safe margin” distance condition. Tissue type sensor is based on electric impedance measurements and NMR response signals invoked by specially designed electro-magnetic pulses. A classification algorithm is used to recognize tissue type based on a previous training step in which many samples of known tissue type were presented to the algorithm. The invention is not specifically related to neural tissues but rather to “tumor” or “healthy” tissue differentiation.

In US 20110098761 (METHOD AND SYSTEM FOR PREVENTING NERVE INJURY DURING A MEDICAL PROCEDURE), A system and method for treating arrythmogenic cardiac tissue regions with a thermal procedure while avoiding hurting the nearby located phrenic nerve. The nerve is located by electrical or magnetical or chemical stimulation, an induced physiological response is measured distally by one of these methods: electromyography, mechanomyography, magnetomyography, end-tidal carbon dioxide measurement, impedance pneumography and a pulse oxymetry.

The first three methods are based on neuromuscular activity and therefore are clearly restricted to motor nerves as their measurement principle relies on the recorded electrical, mechanical or magnetic activity of a connected muscle. This is clearly not applicable to sensory nerves. The last three methods are only indirectly related to the phrenic nerve stimulation, meaning the measured signal is also influenced by several uncontrolled parameters that make it hard to extract the specific contribution of the phrenic nerve stimulation to the overall recorded signal.

In U.S. Pat. No. 5,284,153 (Method for locating a nerve and for protecting nerves from injury during surgery) a method is presented for the localization of a specific peripheral nerve. The localization is performed through an iterative search process in which an electric stimulator, placed at the tip of an injection tool stimulates the tissue at a given location while the potentially evoked neural response is measured with a detecting means placed downstream along the same nerve. The stimulator is iteratively moved until a strong signal is recorded by the detecting means, indicating proximity of the stimulator location to the specific nerve. The same method is proposed for localizing a nerve during surgery in order to avoid damaging it. In this invention, it is implicitly assumed that the detecting means can pick up signal originating from the desired specific same nerve from the beginning of the search process. In practice, this will require to position the detecting means at a location where the specific nerve is known to pass from anatomical prior knowledge and its electric activity clearly measurable. This will usually be the case close to a neuromuscular junction for the specific nerve where, for example, EMG signals can be recorded for the specific nerve. As a consequence, the invention requires stimulation and measurement to be performed at potentially significant distance from each others, with the need to relocate the detection means each time another specific nerve is considered. Moreover, no solution is proposed for sensory or central nerves for which no practically accessible position is usually known a priori for placing the detecting means.

Beside neural sparing, neural detection may also have the purpose of localized denervation. In other words, whereas in the previously discussed prior art neural tissue detection was sought in order to spare the neural tissue, neural tissue detection may be used for the opposite purpose, that is, to detect the neural tissue in order to destroy it. A typical example of this case is the renal sympathetic denervation procedure that is applied in cases where hyperactivity of said nerve causes drugs-treatment resistant (refractory) hypertension.

Noticeably, a system, called “Simplicity” to perform said renal denervation was recently developed by Ardian Ltd (Palo Alto, USA) acquired by Medtronic LTD (USA). In US patent application US20110207758A1, said catheter based method and apparatus are described. Said catheter is guided all the way to the renal artery (left or right) by standard angiographic techniques. When said artery is reached, the tip of the catheter is positioned close to the internal wall of the artery and a radio frequency (RF) energy releasing device, mounted on said catheter's tip, is switched on for a limited amount of time. The procedure is repeated for several positions of the catheter's tip, spaced longitudinally along the renal artery axis and angularly around the same axis, so as to describe a spiral pattern of positions. The released RF doses as well as the number of delivering positions are set empirically.

For the desired purpose of denervation, however, this approach has two main inconveniences. First, the positions at which the energy is released are not necessarily close to a targeted nerve since the actual nerve's position is unknown. This may result in insufficient denervation and poor therapeutic effect. Moreover, since Ardians' Simplicity system is not capable of distinguishing between renal nerve and the renal artery itself, some of the energy releases may hit undesired locations on the renal artery.

Secondly, the amount of energy delivered at each position is constant and does not account for the actual progression of the denervation process. This lack of feedback may result in unnecessarily large energy release, possibly damaging the artery wall, or, alternatively, in insufficient energy release, resulting in poor denervation.

### SUMMARY

Bearing in mind what has been said so far, it is a purpose of this invention to provide a method and apparatus that enhance neural tissue detection by stimulating a targeted area and by measuring possible occurrences of induced response from neural tissue, if such tissue is present in said targeted area and, optionally, enhancing neural tissue sparing and/or neural tissue denervation of said detected neural tissue.

It is an additional purpose of the invention to provide such a method and apparatus whereby the stimulation and/or the detection of neural tissue may be carried out with or without physical contact with the targeted area, according to the specific embodiment of the invention.

It is yet an additional purpose of the invention to provide such an apparatus which may be used in conjunction with a surgical robot or an intravascular catheter, or handheld by the surgeon.

It is stressed that the expressions “in conjunction” or “used in conjunction” means throughout this application any manner in which two or more devices and/or components and/or elements may function together for a certain purpose and said expressions contain, according to the case, also one or more of the following terms and their likes: mounted, integral, attached, communicating, removable, combined, joint, coupled.

It is yet another purpose of the invention to provide such a method and apparatus which is capable of creating in the course of the surgery, when used in conjunction with a surgical robot, a tridimensional mapping of the neural tissue.

It is yet another purpose of the invention to provide such a method and apparatus which is capable, when used in conjunction of an intravascular catheter, of monitoring in real-time the progression of a denervation procedure.

It is a further purpose of the invention to provide an apparatus as mentioned above parts of which may be, in certain embodiments, disposable for practical and hygienic reasons.

It is yet another purpose of the invention to provide a method and apparatus which do not require visual assessment by the medical team for the detection of neural tissue, are fully automated, and may, if so it is desirable, not only notify of the detection of neural tissue but also, additionally or alternatively, influence the functioning of the NTD and/or the functioning of a surgical robot, if the NTD is used in conjunction with such a robot.

It is a further a purpose of the invention to provide a method and apparatus which fulfills the above mentioned purposes while providing real-time assessment of the nature of the tissue which is targeted by the NTD and by taking real-time actions in response to said assessment.

More generally, it is also a purpose of the invention to provide a method and apparatus for the stimulation and detection of neural tissue which can be used in conjunction with any device, either manually or robotically operated, whereby said device is used to perform a surgical procedure.

Additional purposes of the invention will become apparent as the description proceeds.

As previously mentioned in the Field of the Invention, the method and apparatus which are the object of this application will be called hereafter “Neural Tissue Detector” or, in brief, “NTD”.

At the core of this invention is the ability of the NTD to detect neural tissue by stimulating a targeted area and by measuring possible occurrences of induced response from neural tissue, if such tissue is present in said targeted area. The information about the detection of neural tissue may be used for enhancing neural tissue sparing and/or neural tissue denervation of said detected neural tissue, according to the case.

As we have already mentioned before, neural tissue is not easy to detect, among other reasons, because of its size, shape and its being embedded with other tissues.

Therefore, when a surgery is being carried out in an area where there is a potential presence of neural tissue, the NTD searches for said neural tissue as the surgery proceeds.

The search for neural, tissue is based on the possibility of sending impulses to targeted tissues that may contain neural tissue cells. If said neural tissue cells are indeed present in the area targeted by the impulses, the neural tissue cells respond by a change of their membrane electrical potential that follows a characteristic temporal pattern. This pattern is well known in the field of electrophysiology as “action potential” and this term will be used with this meaning in this application.

### BRIEF DESCRIPTION OF THE DRAWINGS

FIG. 1 shows the characteristic temporal pattern of action potential 1, observed at the membrane of a neural cell following stimulation.

FIG. 2 shows a system and apparatus of the Neural Tissue Detector.

FIG. 3 is a very schematic illustration of the main concept which is at the basis of the method used for the NTD 2 functioning.

FIGS. 4A and 4B shown an apparatus in accordance with an embodiment of the present invention.

### DETAILED DESCRIPTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

As used herein, the terms “data,” “content,” “information” and similar terms may be used interchangeably to refer to data capable of being captured, transmitted, received, displayed and/or stored in accordance with various example embodiments. Thus, use of any such terms should not be taken to limit the spirit and scope of the disclosure. Further, where a computing device is described herein to receive data from another computing device, it will be appreciated that the data may be received directly from the another computing device or may be received indirectly via one or more intermediary computing devices, such as, for example, one or more servers, relays, routers, network access points, base stations, and/or the like. Similarly, where a computing device is described herein to send data to another computing device, it will be appreciated that the data may be sent directly to the another computing device or may be sent indirectly via one or more intermediary computing devices, such as, for example, one or more servers, relays, routers, network access points, base stations, and/or the like.

The principles described herein may be embodied in many different forms. Not all of the depicted components may be required, however, and some implementations may include additional, different, or fewer components. Variations in the arrangement and type of the components may be made without departing from the spirit or scope of the claims as set forth herein. Additional, different, or fewer components may be provided.

In order to better understand the interaction between the NTD and neural tissue it may be useful to elaborate on the electrical properties of the neural cells mentioned above. When the membrane's electric potential of a neuron is increased above a “threshold” value (about −55 my), an action potential 1, is observed. During the action potential 1, a depolarization step occurs during which the membrane's polarity changes until a positive peak (around 40 my) is reached. In the course of the following repolarization step, the membrane progressively recovers its negative potential. An undershoot is observed before the initial resting state potential is reached again (refractory period). The action potential 1 propagates from the axon's hillock to the end of the axon (axon terminals) and constitutes the way of transmitting electrical signals between neural cells.

There are a number of points that are worth noticing following this short overview of the electrical properties of the neural cells and the relevance of these properties to the present invention.

First, the magnitude of the electrical activity of said cells is significant enough for electronic measurement purposes. Secondly, the interaction between the NTD and neural tissue is clearly faster and provides more accurate information regarding the presence of neural tissue than what has been described in the prior art. This, because the NTD measures the induced response from the neural tissue in the range of a very small targeted area rather than responses provided by muscles connected to stimulated neural tissue which are usually located at significant distance from the targeted area and are subject to possible signal disturbances.

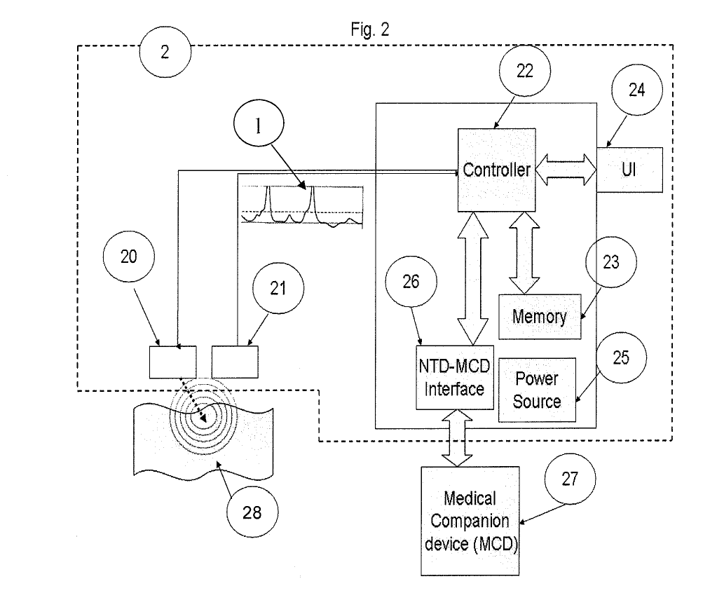

After that having briefly illustrated electrical properties of the nervous tissue cells which are used for creating an interaction between the NTD and the nervous cells, we will illustrate a sample structure and the main components of the NTD apparatus using FIG. 2.

Before we do so, it is stressed that throughout this application any component and/or element described in any of the figures contained in the application may be referred to interchangeably in a variety of ways including: by its full name, in its singular or plural form, followed or not by the number indicating said component or element in a figure, by said number only, by an abbreviation or an acronym of the name—bracketed or non-bracketed—of any of above mentioned component or element, whether or not these are followed b the indicating number or by any combination or variation of any of the previous options and regardless of any typographical feature.

In its basic structure, the apparatus of the Neural Tissue Detector (NTD)—indicated in FIG. 2 by number 2—requires the following components:

1. An emitter 20 capable of generating impulses which excite the neural cells and causes the generation and propagation of action potentials if neural cells are present in the targeted area. The emitter 20, depending on specific embodiments, may be of different types including but not exclusively, any of the following ones: electric, such as an electrode or an array of electrodes, magnetic, mechanical, acoustic, infrared laser, thermal and chemical, whereby a device releases a liquid chemical solution.

2. A receiver that functions as the action potential detector (APD) 21, which detects the action potentials 1 induced in the neurons by the emitter's signal. The APD 21 may be a component capable of measuring membrane potential such as an electrode or an array of electrodes, or any other variable functionally dependent on the membrane potential, including, but not exclusively, current, electric field as can be measured by a high sensitivity whispering gallery mode resonator, magnetic field, thermal emission, optical birefringence response, acoustic and, more generally, any measurable change associated with membrane potential. If desirable, it is possible to integrate the emitter 20 and the APD 21 into the same component. For example the multi-electrode arrays (MEA) systems produced by AxionBiosystems (Atlanta, Ga.) can be used to stimulate and record the response of the neural tissue simultaneously.

Two observations regarding the emitter 20 and the APD 21 are in order:

(a) The emitter 20 and the action potential detector 21 are mounted at a fixed distance and the action potential detector 21 is placed sufficiently close to the area targeted by the emitter 20 as to detect the action potential 1 at close proximity from its generation site.

(b) The emitter 20 and/or the APD 21 may be of a type that requires or does not require physical contact with the targeted area 28.

3. a Controller 22 for running all required software necessary for governing the operation of the NTD 2—hereafter “NTD software”—which is responsible for one or more of the following: elaborating data, carrying out calculations, communicating and issuing commands and signals to NTD 2 components, communicating with any other device which may optionally work in conjunction with the NTD 2, for example a medical companion device (MCD) 27 for instance, a surgical robot and, optionally, influencing the functioning of the MCD 27 and, generating information which will be handled by the MCD-NTD interface 26 and/or by User Interface 24.

4. Memory 23 comprising of one or more memory means of any kind including read/write volatile and/or non-volatile memory such as controller's on-board memory, hard disk drive, memory cards (e.g. flash card), ROM, RAM and/or any kind of memory external to the NTD 2 (e.g. memory means in a surgical robot system if used in conjunction of the NTD 2) or any combination thereof.

5. User Interface (UI) 24 comprising of any suitable I\O means which enable the user to input data or commands of any kind to the NTD 2 and allow the NTD 2 to manifest any kind of information to the external world including acoustic or visual notifications for example, regarding the detection of nervous cells tissue and/or other information originated by the MCD 27.

6. Power Source 25 comprising of an electrical power source of any suitable type for sustaining the functioning of the NTD 2.

It goes without saying that the short description above of the basic structure of the apparatus of the NTD 2 focuses on the inventive aspects of this novel apparatus while, for the sake of conciseness, the description of obvious components or elements is not meant to be exhaustive as these elements would be self-evident for any person skilled in the art.

Accordingly, the short description above of the basic structure of the apparatus of the NTD 2 should be construed as containing any possible variation of said structure which does not change the essence of the invention including, but not exclusively, one or more of the following: different configuration or arrangement of the apparatus components, addition to or omission from the NTD 2 of one or more components, regardless whether said components are known or unknown at the time of the filing of this patent application.

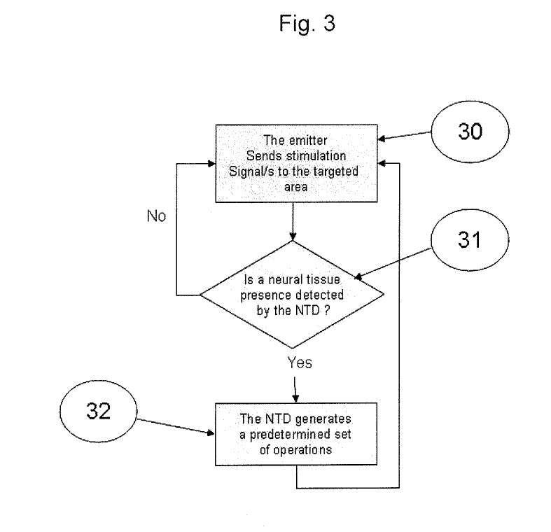

After the general structure of the apparatus of the NTD 2 and its main components have been described, we will proceed, using FIG. 3, with a general description of the method used in the functioning of the NTD 2.

FIG. 3 is a very schematic illustration of the main concept which is at the basis of the method used for the NTD 2 functioning.

As it will be shown hereafter, said method of the invention for the detection of neural tissue in a body's targeted area 28 comprises, in its basic form, of the following steps:

i. Sending one or more impulses generated by the neural tissue detector (NTD) emitter 20 to the targeted area 28 which impulse/s is/are capable of causing nervous tissue, if such tissue is present in the targeted area 28, to respond to the impulse/s by generating and propagating one or more action potential/s 1 and;

ii. Detecting the action potential/s 1 described in (i) by using an action potential detector (APD) 21 mounted at a fixed distance from the emitter 20 and by positioning said APD 21 sufficiently close to the targeted area 28 as to detect the action potential 1 in the immediate vicinity of its generation site and

iii. Running the NTD software which governs all the functioning of the NTD 2 including steps (i) and (ii), optionally acting according to information derived by said steps and;

iv. Generating a predetermined set of operations as a function of the presence of neural tissue in the targeted area 28 and;

v. repeating steps (i) to (iv) as many times as it may be required in the course of the medical procedure

Now, going back to FIG. 3 we will elaborate on the method of the invention. The figure does not show the switching on or off or pausing of the NTD 2 as these capabilities are self-evident and assumed.

FIG. 3 shows at 30 the NTD's emitter 20 sending stimulation signal/s to the targeted area 28. As already mentioned, the stimulation signal sent by the emitter 20 may be of any suitable type like, an IR laser, an acoustic wave, electric or magnetic pulse, etc. The emitters types capable of generating said stimulation signals have already been described above in connection with the NTD 2 apparatus.

If neural tissue presence is not detected by the NTD 2 at 31, a new cycle is repeated at 30.

Now it is appropriate to elaborate on the term “neural tissue presence detection”. It should be noted that the terms “action potential detection” and “neural tissue presence detection” do not necessarily overlap and, actually, in many cases, might differ from each other.

In order to better illustrate this differentiation, let us consider the case in which a single stimulation signal generated by the emitter 20 is followed by an APD 21 reading of what is assumed to be a response induced by said stimulation signal.

Firstly, it should be noted that the assumed response measured by the APD 21 might not be the result of the stimulation signal generated by the emitter 20 but, rather, some kind of interference. For example, such interference might derive from electrical activity of neighboring areas of the targeted tissue 28 but not from the targeted tissue 28 itself. Interferences might also be the result of electromagnetic activity of surrounding devices.

Consequently, a single assumed response measured by the APD 21 does not conclusively points to the detection of neural tissue presence in the targeted area 28.

Furthermore, especially when the NTD 2 is a handheld device held by a surgeon (as opposed to an NTD 2 mounted, for instance, on a robot), there is the possibility that after the APD 21 has measured a response induced by said stimulation signal, and even if said response is a real one, the hand of the surgeon which holds the NTD 2 has shifted to a location which does not correspond any longer to the targeted area 28 in which the neural tissue was detected.

To sum things up, a single assumed response to a simulation signal measured by the APD 21 may not be associated to the presence of neural tissue in the targeted area 28 or may not be relevant to the current position in which the NTD 2 is located after the APD 21 measurement.

In order to ensure that the above mentioned uncertainties are overcome and an action potential 1 detection indicates conclusively an actual neural tissue presence detection, a number of strategies, such, for instance, the use of certain emission and response patterns, will be adopted and described in the following embodiments of the invention.

Now, bearing in mind the above mentioned problems, if the presence of neural tissue is conclusively detected, as shown in 31, then the NTD 2 generates a predetermined set of operations 32 which may vary according to specific embodiments.

It is stressed that the flow of the method described in FIG. 3 is governed by the NTD software.

A few examples of one or more of the operations which may be optionally carried out in response to the detection of the presence of neural tissue in the targeted area 28 are:
an acoustic notification generated by the user interface 24.
a visual notification, such as a blinking light or a textual/graphical message displayed by the user interface 24.
recording of information such as the position of the detected neural tissue in the targeted area 28.

In the case that the NTD 2 is used in conjunction with a MCD 27 one or more of the following options are also possible:
influencing the functioning of the MCD 27 used in conjunction with the NTD 2, for instance, inhibiting the functioning of the surgical tool that is operating on the area targeted 28 by the emitter 20.
recording encoders' positions of a MCD 27—for instance, a surgical robot—whereby said positions correspond to a detected neural tissue position in order to prevent tissue destruction at these recorded locations, optionally making use of stereotactic surgery techniques.
in the case of denervation procedure, releasing an appropriate type and amount of energy in order to destroy the detected neural tissue.
creating a tridimensional mapping of the neural tissue detected in the course of the surgery by recording the accumulated set of encoders' positions and by displaying and/or storing said tridimensional mapping for reference.
Any combination of two or more of the above listed possible operations in response to the detection of the presence of neural tissue in the targeted area 28.

It goes without saying that the short description above focuses on the inventive aspects of the method used with the NTD 2 while, for the sake of conciseness, the description of obvious steps is not meant to be exhaustive as these elements would be self-evident for any person skilled in the art.

Accordingly, the short description above of the method should be construed as containing any possible variation of said method which does not change the essence of the invention including, but not exclusively, one or more of the following: different configuration, sequence, timing or arrangement of steps comprised in the method and/or addition to or omission from the method of one or more steps.

It should also be noticed that, for the purposes of this application, is irrelevant which programming language or environment and/or network or communication protocol are used for the implementation of the method and/or if the steps of the method are implemented by software logic only (that is, by the NTD software), by hardware logic (e.g. by appropriate circuitry) or by a combination of software and hardware logic.

We will now proceed with a detailed description of a number of sample preferred embodiments of the invention. It is stressed that the following preferred embodiments are only a small, non limitative, number of the many possible embodiments of the invention disclosed in this application and are not meant to restrict in any way whatsoever the scope of the invention.

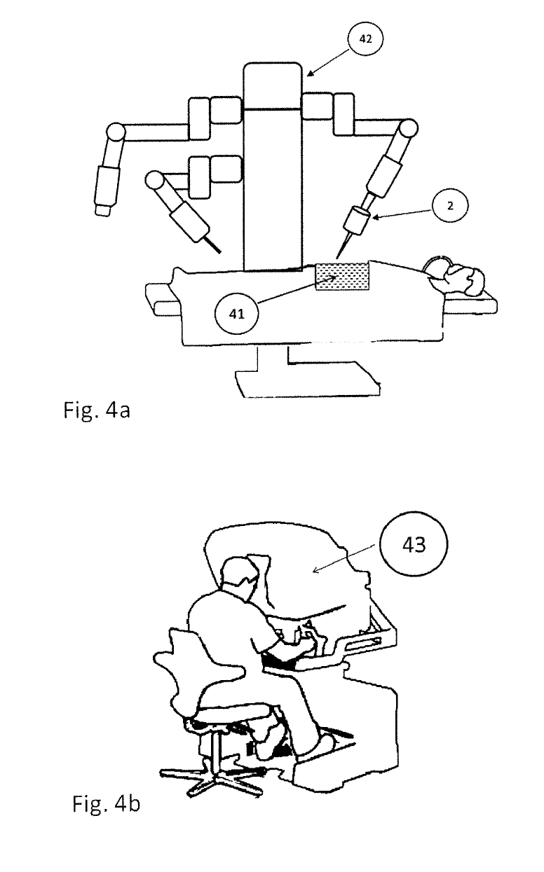

One embodiment based on the invention will be described using FIGS. 4a and 4b. In FIG. 4a, a patient is shown lying on a surgical bed. The surgical field 41 (that is, the area of interest) is the area of the patient's body which is about to be operated. The surgery is going to be carried out using a surgical robot 42 which fulfills, for the purposes of this embodiment, the functions of the MCD 27. Surgical robots are nowadays increasingly being used because of the significant advantages they offer, such as reduced invasiveness, high accuracy and operational speed, which generally translate in faster and easier post-operatory recovery. The robot 42 illustrated in FIG. 4a is a typical surgical robot known as “Da Vinci” (by Intuitive Surgical, Sunnyvale, Calif., USA) on which robot 42 a NTD 2 has been mounted. The NTD 2, in this embodiment may be mounted on robot 42 in a fixed or removable manner, as it may be convenient. The NTD 2 is shown symbolically in FIG. 4a as mounted on one arm of the robot 42. The NTD 2 in FIG. 4a represents all the NTD 2 components that may be required for this specific embodiment including, of course, emitter 20 and APD 21 although the said components are not graphically shown. During surgery, the robot 42 actions and movements are remotely controlled in real time by a surgeon through a remote control cabinet 43 shown in FIG. 4b. The surgical robot 42 and the remote control cabinet 43 and any other hardware and/or software related to the functioning of these elements comprised in this embodiment have been defined in the summary of the invention as the medical companion device MCD 27 which operates in conjunction with NTD 2 which is the object of the invention disclosed in this application. It should be understood that in this embodiment and in all the embodiments which contemplate an NTD 2 which functions in conjunction with a MCD 27, one or more parts of the NTD 2 may be detachable from the MCD 27 or may be an integral part of it or vice-versa. Therefore, the term “mounted” in this application means any kind of temporary or permanent physical contact, that enables the fulfillment of the embodiment. It is also stressed that certain elements of the NTD 2 and/or the MCD 27 may function jointly without physical contact through different means of communication, including wireless communication ones.

Furthermore, one or more parts of the NTD 2 and/or the MCD 27 might be disposable if this should be desirable for hygienic or other practical considerations.

In this embodiment the emitter 20 comprises an IR laser diode which is used in a typical wavelength range of about 1.4 μm to 1.9 μm. Such an IR laser diode is capable of working with pulse duration of about 35 μs to 1000 μs at repetition rate of up to 1000 pulses a second.

The IR laser beam width in this embodiment is of about 10 μm to 200 μm. The advantage in using an emitter of this kind is that no physical contact is required between said emitter 20 and the targeted tissue 28 thus, avoiding damage related to possible contamination, mechanical injury and chemical incompatibility. An additional advantage of using an IR laser beam as an emitter 20 is that the targeted tissue can be stimulated in a very precise manner due to the ability to highly focus the IR laser beam.

In this embodiment the APD 21 consists of a high-sensitivity electric field sensor relying on a whispering gallery mode resonator. A whispering gallery resonator contains a transparent dielectric microsphere the physical dimensions of which are affected by certain influences. As the physical dimension varies the internal optical path of an incident laser beam trapped into the microsphere is modulated resulting in measurable discrete modes (called “whispering gallery modes”). Since the dimensions of a microsphere made of certain materials (e.g. certain polymers) are affected by electric field, a whispering gallery mode resonator may provide a suitable APD 21 for this embodiment. This, because we are interested in measuring the response of nervous tissue to stimulation, where such response consists of an action potential 1 which generates an electric field.

Again, as it was said for the emitter 20, one of the advantages of using this type of electric field sensor is that no contact is required between the APD 21 and the targeted tissue 28.

Now, once the NTD 2 is activated, the emitter 20 begins to send laser pulses to stimulate the targeted tissues 28. The laser pulses are fired according to predetermined rules contained in the NTD software similarly as already mentioned in relation to FIG. 3.

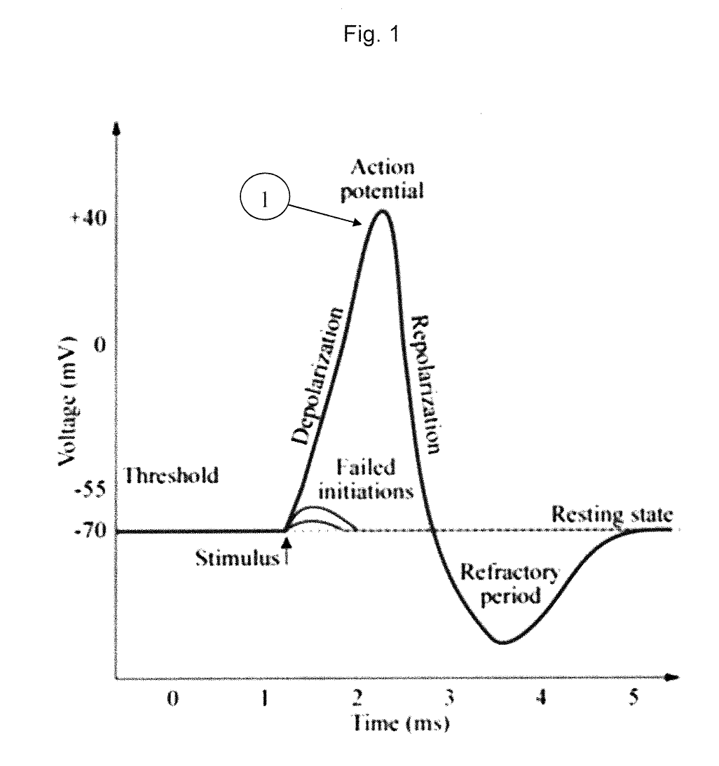

If the laser pulses hit a nervous tissue, an evoked action potential 1 is expected to occur and, accordingly, to generate an electric field as shown previously in connection with FIG. 1.

In order to ensure that the electrical signals picked by the APD 21 are the result of the stimulation exercised on the nervous tissues by the IR laser emitter 20 and not the result of any unrelated physiological activities, or the result of electromagnetic activity of surrounding devices, a certain procedure is adopted.

Such procedure consists of firing the laser pulses on the targeted area 28 according to a certain pattern the variables of which are the number of pulses and time interval between each pulse. These patterns may be of any suitable form consisting, in the simplest case of a single pulse and in other cases of a number of pulses which are timed at fixed or changeable intervals. The patterns may be preprogrammed or automatically generated, whichever is more suitable for the desired result. A pattern of pulses fired by the emitter 20 will be called hereafter “emission pattern”.

In response to the emission pattern the APD 21 measures some electrical signals as explained above. These signals will be called “response pattern”. Similarly to the emission patterns, the response patterns measured by the APD 21 are also defined by number\time variables. In the simplest case a response pattern consists of a single signal.

The NTD software, compares the pattern of the IR laser pulses (emission pattern) with the pattern of the electrical signals picked by the APD 21 (response pattern). If the emission pattern and the response pattern are found to correlate sufficiently, according to predetermined criteria contained in the NTD software, the NTD 2 assumes that a nervous tissue has been found in the area targeted 28 by the emitter 20. If the emission pattern and the response pattern do not correlate sufficiently the emitter 20 keeps firing IR laser pulses, that is, it keeps looking for nervous tissue as shown in FIG. 3.

If nervous tissue is detected, the NTD 2 may optionally generate a predetermined set of operations as previously mentioned in the Summary of the Invention.

Said set of operations may include:

(i) an acoustic notification generated by the User Interface 24.

(ii) a visual notification, such as a blinking light or a textual/graphical message displayed by the user interface 24.

(iii) recording of information such as the position of the detected neural tissue in the targeted area 28.

In the case that the NTD 2 is used in conjunction with a MCD 27 one of the following actions are also possible:

(iv) influencing the functioning of the MCD 27 (e.g. surgical robot 42) used in conjunction with the NTD 2, for instance, by inhibiting the functioning of the surgical tool which is comprised in the MCD 27.

(v) recording encoders' positions of the MCD 27, whereby said positions correspond to a detected neural tissue position in order to prevent tissue destruction at these recorded locations, optionally making use of stereotactic surgery techniques.

(vi) creating a tridimensional mapping of the neural tissue detected in the course of the surgery by recording the accumulated set of encoders' positions and by displaying and/or storing said tridimensional mapping for reference.

While operations (i) to (ii) and (v) to (vi) have already been mentioned in the description, the following additional clarifications in relation with operations (iii) to (vi) may be useful.

Operation (iii) allows the mapping of detected nervous tissues. This carries at least two major advantages. Firstly it will prevent the MCD 27 (e.g. surgical robot 42) from returning to an already detected nervous tissue area and this will result in time saving which, as already mentioned, has important consequences on the surgical procedure.

Secondly, this may help predict the path of the nerves which belongs to the detected areas thus, giving priority to areas which are not yet deemed to contain nervous tissue. This has in its turn two advantages. The first is, again, time saving. The second is to exercise increased caution in an area which is predicted to contain nervous tissue even if a single measurement has not detected it.

In other words, since no system is completely error proof, if the accumulated evidence in the course of the surgery points out to the likelihood that a nervous tissue could be present in a certain area, the NTD 2 will check again said area—possibly by increasing certain sensitivity parameters. For example, stimulation may be provided at progressively increasing intensity of the laser light pulses separated by measurement cycles. If a response signal is recorded at a given stimulation intensity, the intensity level is not further increased.

Operation (iv) may automatically inhibit the action of the surgical portion of surgical robot 42 when the NTD 2 detects the presence of nervous tissue. Of course, the NTD 2 may afford the surgical robot 42 the option to override this operation or, in other words, to perform a surgical action on a certain area if the surgeon wishes to do so in spite of the detection of nervous tissue.

It is important to mention that, according to different variations of this embodiment, the NTD 2 and the MCD 27—in this case, surgical robot 42—may entail a different degree of cooperation, ranging from two separate devices which communicate through an NTD—MCD Interface 26, capable of allowing all the necessary communication between the two devices, to a system in which the NTD 2 and the MCD 27 are fully integrated. In any case, it is stressed than one or more elements of the NTD 2 may be substituted by using the resources of the MCD 27 which, by nature, it is likely to already be equipped with. Thus, in order to avoid unnecessary duplication of components and resources. For instance, the user interface of the NTD 2 may be implemented using the display of the surgical robot's remote control cabinet 43. It goes without saying that the same applies to other NTD 2 components such as controller 22, memory means 23 and power supply 25, which are generally already present and more powerful in the MCD 27 and therefore need not be duplicated in the NTD 2. The same considerations apply, of course, also to software components and, in general, to any instance where the NTD 2 may use MCD 27 resources and vice-versa.

A further embodiment of the invention is a variation of the and may make use of some or more of the software and hardware elements of the first embodiment with the difference that both emitter 20 and APD 21 of the NTD 2 are in physical contact with the targeted tissue 28. This difference has significant implications that will be explained hereafter.

In this second embodiment the emitter 20 consists of at least one stimulating electrode that injects constant current pulses, typically in the range of 1 mA to 10 mA, at a rate of 60 pulses per second. An example of such an emitter is the Ojemann Stimulator manufactured by Radionics Sales Corp, USA. The stimulating electrode may be of mono or bipolar type or of any other suitable type.

The APD 21—that is, the receiver—in this embodiment consists of at least one recording electrode of one of the many well known types. The electrode(s) picks-up the voltage signal evoked by the stimulation of nervous tissues by the emitter 20 which cause the propagation of an action potential 1.

Both these kinds of emitters 20 and receivers 21 are well known in the field of electroneurophysiology.

While this embodiment requires contact and, therefore, does not have certain advantages of the first embodiment, it relies on simpler and more standard techniques. Furthermore, this embodiment is less likely to be influenced by electrical activity unrelated to the emitter 20 stimulation because of the vicinity between the targeted area 28 and the APD 21.

Consequently, the NTD software routines run in order to verify the matching between the emission pattern and the response pattern are also likely to be simplified or even unnecessary. More importantly, because of the contact between the APD 21 and the targeted area 28 the electrical signal picked up by the APD 21 is bound to be stronger and less subject to noise. Of course, as already mentioned before, the trade off for the physical contact involved in this embodiment, is a higher risk or contamination, mechanical risk of injury and biochemical incompatibility between the targeted area 28 and the component/s in contact with the same, in comparison with the first embodiment.

### Third Exemplary Embodiment

A third embodiment comprises a variation of the first or the second embodiment.

While in each of the previous embodiments the emitter 20 and the APD 21 require both physical contact with the targeted area 28 or lack thereof, in the third embodiment, the emitter 20 may require physical contact as in the second embodiment and the APD 21 may be such as not involving physical contact with the targeted area 28 or vice-versa. Apart from the mixed typology of the emitter 20 and the APD 21, this third preferred embodiment is similar in the other aspects to the first and/or the second embodiments.

### Fourth Exemplary Embodiment

In this fourth embodiment, the NTD 2 may be mounted on a surgical tool which is manually held and used by the surgeon without the mediation of a robot or any similar device. It should be noted that although the medical companion device (MCD) in previous embodiments was illustrated using a surgical robot, the MCD 27 may also be a non-robotic manually operated device or system and, more generally, any appropriate surgical tool. This consideration regarding the MCD 27 applies to all embodiments of the invention.

Typical surgical tools that fall in the category of MCD 27 in this embodiment are: scalpels, scissors, electrosurgical forcipes, ultrasonic surgical dissector and aspirator and syringes. Said tools may also optionally be used in a laparoscopic setting.

In this embodiment, as in the previous ones, the NTD 2 may be combined with a manually held surgical tool in any suitable manner which is desirable for a specific implementation of the invention. The NTD 2 may be mounted, attached or coupled or removable from or, integrated with, a surgical tool with which it is used in conjunction in any fashion whatsoever. Moreover, when one of said tools is normally equipped with certain hardware and/or software components, said components may be shared by the surgical tool and the NTD 2 in order to avoid components' duplication in the two devices.

In the present embodiment, the method and apparatus for detecting neural tissue are essentially the same ones as one or more of the previous embodiments with the difference that in this embodiment, as already mentioned, the surgical tools are manually operated by the surgeon.

When a neural tissue is detected, the NTD 2 is capable, as in the previous embodiments, to generate a warning to the surgeon.

However, the ability of the NTD 2 to influence the functioning of the surgical tools depends on the specific surgical tool used in conjunction with the NTD 2. For instance, if the surgical tool in question is a simple scalpel, the operation of which depends solely on the motion of the surgeon's hand, the NTD 2 will not be able to stop automatically the incision produced by the scalpel. On the other hand in the case of an electrosurgical forcipes, the NTD 2, upon detection of neural tissue, can inhibit the functioning of the electrosurgical forcipes even if the surgeon has mistakenly tried to activate the electrosurgical forcipes, for instance, by pressing a foot switch that allows electric current and causes thermal destruction of the targeted tissue 28. In this case, the NTD 2 may simply interrupt the electrical flow into the electrosurgical forcipes. It goes without saying that, optionally, after having inhibited the potentially dangerous surgical action, and after having notified the surgeon, the NTD 2 may be programmed as to enable the surgeon to override the NTD 2 stoppage of the electrical flow and to carry on with the surgical step if the surgeon deems this to be desirable in spite of the detection of neural tissue.

### Fifth Exemplary Embodiment

The fifth embodiment of this invention relates to a NTD 2 which is meant solely to detection of neural tissue and which constitutes a standalone device. In this case, the NTD 2 serves only the purpose of detecting the presence of neural tissue in the course of a surgery but it is not mounted or combined or otherwise used in conjunction, and does not communicate or affect in any manner whatsoever the functioning of any medical surgical tool—that is an MCD 27—that the surgeon uses in the course of the operation and does not interact in any way with the MCD 27.

Apart from the fact of being physically and functionally detached from the MCD 27, the NTD 2 in this embodiment may retain some or all of the NTD 2 functionalities described in the previous embodiments, as it may be advantageous for specific medical requirements.

### Sixth Exemplary Embodiment

This embodiment refers to the case wherein neural detection is performed for the purpose of denervation.

In this embodiment, the NTD 2 is mounted on a medical companion device (MCD) which in this case is a system comprising an intra-vascular catheter on the tip of which is mounted an RF energy releasing source. An example of this kind of systems is Ardian's Simplicity system discussed before in this application in relation to the prior art.

In this embodiment, by mounting the NTD 2 on the MCD 27—the Ardian's Simplicity system or a similar one—the RF energy source located at the catheter tip can be released after the presence of neural tissue in proximity of the renal nerve is established. In other words, first the NTD 2 checks for a response to the stimulation generated by the NTD 2 in the vicinity of the catheter tip and only if such presence is established the catheter tip releases the RF energy required to cause the renal nerve's ablation. Thus, the process of denervation ceases to be an empiric process based on an element of guessing and becomes a closed loop process based on accurate measurement.

This mode of operation holds significant advantages since it enables optimization in terms of amount of released energy, number of energy releasing instances and accuracy in targeting accuracy. Furthermore, the success of the denervation process can be assessed in real time, as the operation proceeds and, therefore, the denervation process may be repeated or shortened in a flexible manner.

Referring back to FIG. 2, the controller may include one or more modules configures to carry out the above described operations and/or steps. As referred to herein, “module” includes hardware, software and/or firmware configured to perform one or more particular functions. In this regard, the means of circuitry as described herein may be embodied as, for example, circuitry, hardware elements (e.g., a suitably programmed processor, combinational logic circuit, and/or the like), a computer program product comprising computer-readable program instructions stored on a non-transitory computer-readable medium (e.g., memory) that is executable by a suitably configured processing device (e.g., processor), or some combination thereof.

In one embodiment, the controller may include or be associated with a processor. The processor may, for example, be embodied as various means including one or more microprocessors with accompanying digital signal processor(s), one or more processor(s) without an accompanying digital signal processor, one or more coprocessors, one or more multi-core processors, one or more controllers, processing circuitry, one or more computers, various other processing elements including integrated circuits such as, for example, an ASIC (application specific integrated circuit) or FPGA (field programmable gate array), or some combination thereof. The controller may include a single processor, or in some embodiments, may comprise a plurality of processors. The plurality of processors may be embodied on a single computing device or may be distributed across a plurality of computing devices collectively configured to function as circuitry. The plurality of processors may be in operative communication with each other and may be collectively configured to perform one or more functionalities of circuitry as described herein. In an example embodiment, processor is configured to execute instructions stored in memory or otherwise accessible to processor. These instructions, when executed by processor, may cause circuitry to perform one or more of the functionalities of circuitry as described herein.

Whether configured by hardware, firmware/software methods, or by a combination thereof, the processor may comprise an entity capable of performing operations according to embodiments of the present invention while configured accordingly. Thus, for example, when processor is embodied as an ASIC, FPGA or the like, processor may comprise specifically configured hardware for conducting one or more operations described herein. As another example, when processor is embodied as an executor of instructions, such as may be stored in memory, the instructions may specifically configure processor to perform one or more algorithms and operations described herein.

Memory 23 may comprise, for example, volatile memory, non-volatile memory, or some combination thereof. Although illustrated in FIG. 2 as a single memory, memory 23 may comprise a plurality of memory components. The plurality of memory components may be embodied on a single computing device or distributed across a plurality of computing devices. In various embodiments, memory 23 may comprise, for example, a hard disk, random access memory, cache memory, flash memory, a compact disc read only memory (CD-ROM), digital versatile disc read only memory (DVD-ROM), an optical disc, circuitry configured to store information, or some combination thereof. Memory 23 may be configured to store information, data, applications, instructions, or the like for enabling circuitry to carry out various functions in accordance with example embodiments discussed herein. For example, in at least some embodiments, memory 23 is configured to buffer input data for processing by processor 22. Additionally or alternatively, in at least some embodiments, memory 23 may be configured to store program instructions for execution by processor 22. Memory 23 may store information in the form of static and/or dynamic information. This stored information may be stored and/or used by circuitry during the course of performing its functionalities.

In some embodiments, the apparatus may include a communications module that may be embodied as any device or means embodied in circuitry, hardware, a computer program product comprising computer readable program instructions stored on a computer readable medium (e.g., memory 23) and executed by a processing device (e.g., processor 22), or a combination thereof that is configured to receive and/or transmit data from/to another device, such as, for example, a second circuitry and/or the like. In some embodiments, communications module (like other components discussed herein) can be at least partially embodied as or otherwise controlled by processor 22. In this regard, a communications module may be in communication with processor 22, such as via a bus. Communications module may include, for example, an antenna, a transmitter, a receiver, a transceiver, network interface card and/or supporting hardware and/or firmware/software for enabling communications with another computing device. Communications module may be configured to receive and/or transmit any data that may be stored by memory 22 using any protocol that may be used for communications between computing devices. Communications module may additionally or alternatively be in communication with the memory 22, an input/output module and/or any other component of circuitry, such as via a bus.

User Interface module may be in communication with processor 22 to receive an indication of a user input and/or to provide an audible, visual, mechanical, or other output to a user. Some example visual outputs that may be provided to a user by circuitry are discussed in connection with the displays described above. As such, the user interface may include/comprise an input/output module and may include support, for example, for a keyboard, a mouse, a joystick, a display, an image capturing device, a touch screen display, a microphone, a speaker, a RFID reader, barcode reader, biometric scanner, and/or other input/output mechanisms. In embodiments wherein circuitry is embodied as a server or database, aspects of input/output module may be reduced as compared to embodiments where circuitry is implemented as an end-user machine (e.g., consumer device and/or merchant device) or other type of device designed for complex user interactions. In some embodiments (like other components discussed herein), input/output module may even be eliminated from circuitry. Input/output module 908 may be in communication with memory, communications module, and/or any other component(s), such as via a bus.

In some embodiments, the system may also include a MCD-NTD interface module 26 that may also or instead be included and configured to perform the functionality discussed herein related to data transmitted between the NTD and MCD.

In some embodiments, some or all of the functionality facilitating control of the MCD, NTD, and analysis of the data may be performed by processor 22. For example, non-transitory computer readable storage media can be configured to store firmware, one or more application programs, and/or other software, which include instructions and other computer-readable program code portions that can be executed to control processors of the components of system to implement various operations, including the examples shown above. As such, a series of computer-readable program code portions may be embodied in one or more computer program products and can be used, with a computing device, server, and/or other programmable apparatus, to produce the machine-implemented processes discussed herein.

Any such computer program instructions and/or other type of code may be loaded onto a computer, processor or other programmable apparatuses circuitry to produce a machine, such that the computer, processor other programmable circuitry that executes the code may be the means for implementing various functions, including those described herein.

The illustrations described herein are intended to provide a general understanding of the structure of various embodiments. The illustrations are not intended to serve as a complete description of all of the elements and features of apparatus, processors, and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

The above disclosed subject matter is to be considered illustrative, and not restrictive, and the appended claims are intended to cover all such modifications, enhancements, and other embodiments, which fall within the true spirit and scope of the description. Thus, to the maximum extent allowed by law, the scope is to be determined by the broadest permissible interpretation of the following claims and their equivalents, and shall not be restricted or limited by the foregoing detailed description. While various preferred embodiments of the invention have been described in this application, it is stressed that these embodiments are meant as a limited and non-exhaustive number of examples of possible embodiments of the invention and that many other embodiments of the invention are possible without departing from the spirit and scope of the invention as described and claimed in this patent application.

1. Method for the detection of neural tissue in a body's targeted area which consists of the following steps:
i. sending one or more impulses generated by the neural tissue detector (NTD) emitter to the targeted area which impulse/s is/are capable of causing nervous tissue, if such tissue is present in the targeted area, to respond to the impulse/s by generating and propagating one or more action potential/s and;
ii. detecting the action potential/s described in (i) by using an action potential detector (APD) mounted at a fixed distance from the emitter and by positioning said APD sufficiently close to the targeted area as to detect the action potential in the immediate vicinity of its generation site and;
iii. running the NTD software which governs all the functioning of the NTD including steps (i) and (ii), optionally acting according to information derived by said steps and;
iv. generating a predetermined set of operations as a function of the presence of neural tissue in the targeted area and;
v. repeating steps (i) to (iv) as many times as it may be required in the course of the medical procedure.

2. Method according to claim 1, wherein the emitter requires a physical contact with the targeted tissue and is either a single contacting electrode or an array of contacting electrodes.

3. Method according to claim 1, wherein the emitter does not require a physical contact with the targeted tissue and is of one of the following kinds:
a IR laser diode, an electric or magnetic field generator or an acoustic wave generator.

4. Method according to claim 1, wherein the action potential detector (APD) requires a physical contact with the target tissue and is either a single contacting electrode or an array of contacting electrodes.

5. Method according to claim 1 wherein, the APD does not require a physical contact with the target tissue and is of one of the following kinds:
a high-sensitivity electric field sensor and, in particular, but not exclusively, such a sensor relying on a whispering gallery mode resonator, a high-sensitivity magnetic field sensor, an optical birefringence response sensor or any other suitable sensor including a thermal sensor.

6. Method according to claim 1, wherein the medical instruments combined with the NTD are manually operated and are among the following kinds:
scalpels, scissors, electrosurgical forcipes, ultrasonic surgical dissector and aspirator, syringes whether said tools are used either in a conventional or laparoscopic setting.

7. Method according to claim 1, wherein the NTD is combined in any manner with a surgical robot including being completely or partially integral to the surgical robot or fully removable from the same.

8. Method according to claim 7, wherein encoders' coordinates of a surgical robot (MDC) corresponding to detected neural tissue positions are recorded to create incrementally a tridimensional mapping of the neural tissue positions as the surgery proceeds.

9. Method according to claim 8, wherein the incrementally created tridimensional mapping of the detected neural tissue is used in any one or more of the following ways: stored in a volatile and/or non-volatile memory means, displayed in any manner, transmitted in any manner to a device comprised or external to the NTD or the NTD environment or to a remote location, manipulated in any manner meant to derive new data or images, interpolating or extrapolating neural tissue mapping.

10. Method according to claim 1, wherein in order to ensure that the electrical signals picked by the APD are the result of the stimulation exercised on the nervous tissues by the emitter and not the result of any unrelated physiological activities, an emission pattern consisting of signals fired by the emitter is compared to a response pattern consisting of signals measured by the APD.

11. Method according to claim 1 wherein upon detection of neural tissue or after processing data related to said detection, the NTD responds in any suitable manner including, but not exclusively, in one or more of the following manners: an acoustic notification generated by the user interface, a visual notification, such as a blinking light or a textual/graphical message displayed by the user interface.

12. Method according to any of the previous claims, wherein upon detection of neural tissue or after processing data related to said detection, the NTD responds by influencing the functioning of the MCD used in conjunction with the NTD, for instance, by inhibiting or enabling the functioning of the surgical tool that is operating on the targeted area based on detection of neural tissue or absence thereof.

13. A Neural Tissue Detector apparatus (NTD) for the detection of neural tissue in a body's targeted area comprising:
i. An emitter capable of generating impulses which excite the neural cells and causes the generation and propagation of action potentials if neural cells are present in the targeted area and;
ii. an action potential detector (APD), for the detection of the action potentials described in (i) being said APD mounted at a fixed distance from the emitter and;
iii. a Controller which is responsible for governing the operation of the NTD, including running the NTD software, and communicating with and/or influencing the functioning of any other device which may optionally work in conjunction with the NTD, including a medical companion device (MCD).
iv. one or more memory means of any kind of memory internal or external to the NTD and/or any combination thereof;
v. User Interface—any I\O means which enable the user to input data or commands to the NTD and allow the NTD to manifest said information to the external world including in any suitable manner;
vi. an Electrical Power Source of any suitable type for sustaining the functioning of the NTD and
vii. optionally an interface for connecting the NTD with the MCD in order to enable the NTD to influence the functioning of the MCD and/or using its resources in any manner and/or, if desirable, vice versa.

14. Apparatus according to claim 13, wherein the detection of neural tissue is used for enhancing neural tissue sparing.

15. Apparatus according to claim 13, wherein the detection of neural tissue is used for enhancing denervation.

16. Apparatus according to claim 13, wherein the emitter requires a physical contact with the targeted tissue and is either a single contacting electrode or an array of contacting electrodes or a device which releases a liquid chemical solution.

17. Apparatus according to claim 13, wherein the emitter does not require a physical contact with the target tissue and is of one of the following kinds:
an IR laser diode, an electric or magnetic field generator, an acoustic wave generator, a thermal power generator or a device which releases a liquid chemical solution which solution, but not the emitter itself, comes in contact with the targeted tissues.

18. Apparatus according to claim 13, wherein the action potential detector (APD) does not require a physical contact with the targeted tissue and may be one or more of the following kinds: a high-sensitivity electric field sensor and, in particular, but not exclusively, such a sensor relying on a whispering gallery mode resonator, a high-sensitivity magnetic field sensor, an optical birefringence response sensor, an acoustic sensor, or any other suitable sensor including a thermal emission sensor.

19. Apparatus according to claim 13, wherein the action potential detector (APD) requires a physical contact with the targeted tissue and is either a single contacting electrode or an array of contacting electrodes.

20. Apparatus according to claim 13, wherein the emitter and the action potential detector are in a single component or consist of a component capable of functioning both as emitter and action potential detector.

21. Apparatus according to claim 13, wherein one or more parts of the NTD are disposable parts.
